(19) United States
(12) Patent Application Publication
(10) Pub. No.: US 2020/0352503 A1
(43) Pub. Date: Nov. 12, 2020

(54) SLEEP APNEA DETECTION

(71) Applicant: Jason Charles Browne, Portola Valley, CA (US)

(72) Inventor: Jason Charles Browne, Portola Valley, CA (US)

(73) Assignee: Jason Charles Browne, Portola Valley, CA (US)

(21) Appl. No.: 16/868,378

(22) Filed: May 6, 2020

Related U.S. Application Data
(60) Provisional application No. 62/844,672, filed on May 7, 2019.

(51) Int. Cl. A61B 5/00; A61B 5/113; A61B 5/11

(52) U.S. Cl. CPC A61B 5/4818; A61B 5/113; A61B 5/1116; A61B 5/6833; A61B 2560/0209; A61B 5/002; A61B 2562/0219; A61B 2562/028; A61B 5/684

(57) ABSTRACT

The present disclosure teaches systems, devices, and methods to detect sleep apnea. A method, for example, includes affixing a sleep apnea detection device to a patient. The sleep apnea detection device has a processor, at least one memory, at least one micro-electromechanical system (MEMs) device, at least one transceiver, and at least one power source to power the sleep apnea detection device. Data that indicates the patient's breathing is collected with the at least one MEMs device, and based on the collected data, at least one of the patient's inhalations and the patient exhalations is determined. It is also determined when the patient fails to inhale over a determined first period of time. Based on a number of times the patient fails to inhale, an Apnea Hypopnea Index (AHI) score is calculated, and if the AHI score crosses a selected threshold, it is determined that the patient has sleep apnea.

### BACKGROUND

### Technical Field

The present disclosure generally relates to systems, devices, and methods associated with sleep apnea. More particularly, but not exclusively, the present disclosure relates to detection of sleep apnea.

### Description of the Related Art

According to the World Health Organization, sleep apnea is a medical condition that affects up to 6% of adults and 2% of children. One reason why an accurate count of the number of sleep apnea sufferers does not exist is because there are no inexpensive, simple, and reliable ways to properly diagnose sleep apnea. This absence of accurate information may cause a large number of people to go without important treatment, which can both help them get better sleep and help them live longer, happier lives. Conversely, some patients are improperly diagnosed with apnea, and these people may be working to remediate a problem that they do not have.

Conventionally, when a medical practitioner suspects that a patient has sleep apnea, the medical practitioner will order a “sleep study.” The sleep study is generally performed in a medical facility where the patient sleeps in a hospital-style or hotel-style bed for at least one night. The sleep study environment can be disruptive to a patient's normal sleep patterns, and so the sleep study may provide less than ideal data. Nevertheless, the sleep study is the current state of technology to determine sleep apnea. In the sleep study, in addition to sleeping in a different-than-normal environment, the patient is also expected to sleep with ten to twenty electrodes adhered to different locations on their body. The electrodes, which are typically attached to the patient's scalp, chest, legs and back provide data to obtain a polysomnography (PSG). The process to affix the electrodes to the patient takes about one hour.

During the sleep study, after the electrodes are placed and wired to a computer, the patient lies down on the hospital/hotel bed and attempts to sleep for up to eight hours. During sleep, despite the new environment and restricted freedom of movement, PSG data is collected.

PSG data can be very helpful to a medical practitioner. For example, the PSG data can be used to determine the exact type of sleep apnea a patient might be suffering from. On the other hand, due to the disruption in the patient's normal sleep habit, many medical practitioners recognize that the absolute level of sleep apnea, as measured with an apnea hypopnea index (AHI) score, may not be completely accurate or repeatable. For these reasons, it is often accepted that the severity of sleep apnea detection by conventional methods is not very accurate, and at least in some cases, medical practitioners judge whether or not the patient actually needs treatment in the form of a CPAP machine without fully accurate information.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

The following is a summary of the present disclosure to provide an introductory understanding of some features and context. This summary is not intended to identify key or critical elements of the present disclosure or to delineate the scope of the disclosure. This summary presents certain concepts of the present disclosure in a simplified form as a prelude to the more detailed description that is later presented.

The device, method, and system embodiments described in this disclosure (i.e., the teachings of this disclosure) enable a medical practitioner or an individual patient to determine if the patient has sleep apnea using a minimally invasive electronic device arranged for a specific purpose. Using data from one or more micro-electromechanical system (MEM's) devices, a patient's paused breathing events during a sleep session can be identified with confidence and counted over any determined time period. The collected and determined information can be used to determine whether or not the patient has sleep apnea, and if so, the level of severity of the sleep apnea.

This Brief Summary has been provided to introduce certain concepts in a simplified form that are further described in detail below in the Detailed Description. Except where otherwise expressly stated, the Brief Summary does not identify key or essential features of the claimed subject matter, nor is it intended to limit the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a sleep apnea detection environment embodiment;

FIG. 2A is sleep apnea detection device embodiment;

FIG. 2B is an embodiment of the sleep apnea detection device embodiment of FIG. 2A with the flexible covering removed;

FIG. 3 is an electronics module embodiment of a sleep apnea detection device;

FIGS. 4A and 4B are sleep apnea detection device embodiments;

FIG. 5 is a right side, cross-sectional view of yet one more sleep apnea detection device embodiment;

FIG. 6 is a system that communicatively couples a sleep apnea detection device to a computing device;

FIG. 7 is a graph representing accelerometer data of a 48 year old man breathing in a supine position on a bed; and

FIG. 8 is a data flow diagram representing a sleep apnea detection process carried out with a sleep apnea detection device embodiment.

### DETAILED DESCRIPTION

The present invention may be understood more readily by reference to this detailed description of the invention. The terminology used herein is for the purpose of describing specific embodiments only and is not limiting to the claims unless a court or accepted body of competent jurisdiction determines that such terminology is limiting. Unless specifically defined herein, the terminology used herein is to be given its traditional meaning as known in the relevant art.

In the present case, the inventor has recognized that misdiagnosis of sleep apnea can cause one set of problems, and the misdiagnosis of no-sleep-apnea can cause a different set of problems. Sleep apnea is treated with a continuous positive airway pressure (CPAP) machine. A CPAP machine is expensive, invasive, and typically uncomfortable for a patient to use. Nevertheless, a CPAP machine remains the accepted form of non-surgical treatment currently available for sleep apnea sufferers. A patient that is misdiagnosed as having sleep apnea bears the expense and inconvenience of a CPAP machine, and a patient misdiagnosed as not having sleep apnea when they in fact do may suffer severe health consequences. Hence, systems, devices, and methods (i.e., the teaching) of the present disclosure to more accurately determine sleep apnea, and the teaching to accurately determine sleep apnea in an environment that is comfortable to the patient provide a valuable solution to society. In addition, because of the challenges to administer a sleep study, the inventor has learned that children and adolescents are seldom diagnosed with sleep apnea. The present teaching is applicable to patients of all ages, sizes, physical capabilities, and other such characteristics.

The device, method, and system embodiments described in this disclosure (i.e., the teachings of this disclosure) represent a practical application of technology to determine whether or not a patient has sleep apnea. In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with computing systems, including client and server computing systems as well as networks, have not been shown or described in detail to avoid unnecessarily obscuring descriptions of the embodiments.

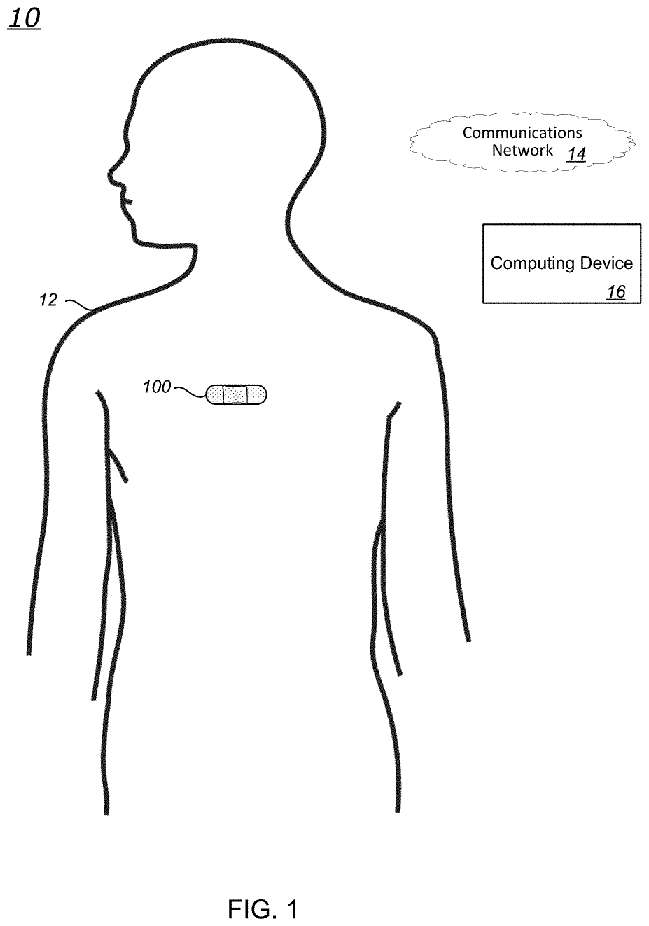

FIG. 1 is a sleep apnea detection environment embodiment 10. In the embodiment, a patient 12 has a sleep apnea detection device 100 temporarily affixed in a selected orientation and in a selected position on his torso. The selected position may be in the middle of the chest, above the diaphragm, above the sternum, in the general proximity of the thoracic cavity, or in some other selected location of the abdomen wherein the sleep apnea detection device 100 is subject to motion caused by the patient's breathing. In at least some optional embodiments, the patient is in the environment of a computing network 14, which is communicatively coupled or coupleable to a computing device 16. After the sleep apnea detection device 100 is located on the patient's body, the patient will go to sleep. The patient may preferably sleep in his own bed and according to his own schedule, but many other optional arrangements are contemplated such as in a selected sleep environment, in a selected bed, in the presence of a medical practitioner, and the like.

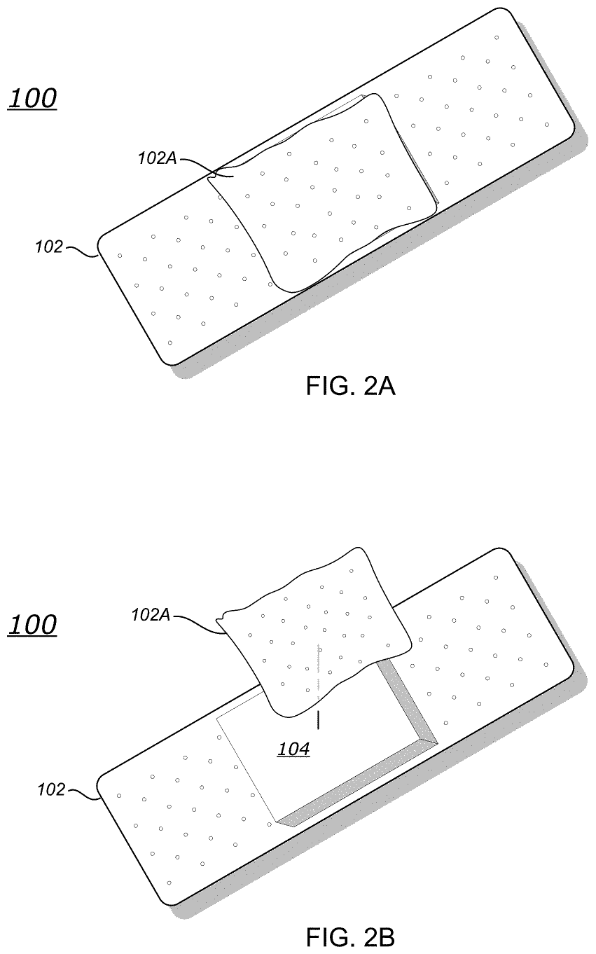

FIG. 2A is an embodiment of a sleep apnea detection device 100 embodiment in more detail. The sleep apnea detection device 100 resembles a conventional adhesive bandage with apertures for air and moisture flow and to provide flexibility. Other form factors are contemplated. In the embodiment of FIG. 2A, a flexible substrate 102 is arranged with a skin-safe adhesive applied on one side (not shown). A central portion of the sleep apnea detection device 100 includes a flexible covering 102A, which may be formed of the same material as the flexible substrate 102. The flexible substrate 102 may be formed from plastic, silicon, silicone rubber, a thin tape, nylon, cotton fabric, or some other flexible material. The flexible substrate 102 has an adhesion index and a flexibility index selected for patient comfort, temporary affixation sufficient to last through a night of sleep, anti-allergen properties, and a reasonable, pain-free or pain-limited removal.

FIG. 2B is an embodiment of the sleep apnea detection device 100 embodiment of FIG. 2A with the flexible covering 102A removed. The flexible covering 102A in some embodiments is integrated with the flexible substrate 102 and not removable. FIG. 2B is arranged to illustrate the inventive teaching herein and one of skill in the art will recognize many other embodiments.

In FIG. 2B, removal of the flexible covering 102A exposes an electronics module 104. The electronics module 104 is represented as formed in a discrete housing, but in some cases, no such housing is present. In some cases, electronic structures of the electronics module 104 are formed directly on a flexible substrate 102 or in some other form factor.

In FIGS. 2A-2B, the sleep apnea detection device 100 is sized about three inches long by one inch wide by one quarter inch in depth (i.e., 3 in.×1 in.×0.25 in.). In other cases, the sleep apnea detection device 100 is sized about three quarters of a cubic inch (i.e., 0.75 in³). In still other cases, the sleep apnea detection device 100 has a longest dimension of about eight inches or less, a longest dimension of four inches or less, or even a longest dimension of about 2 inches or less. In some cases, the sleep apnea detection device 100 has a height of less than one quarter inch.

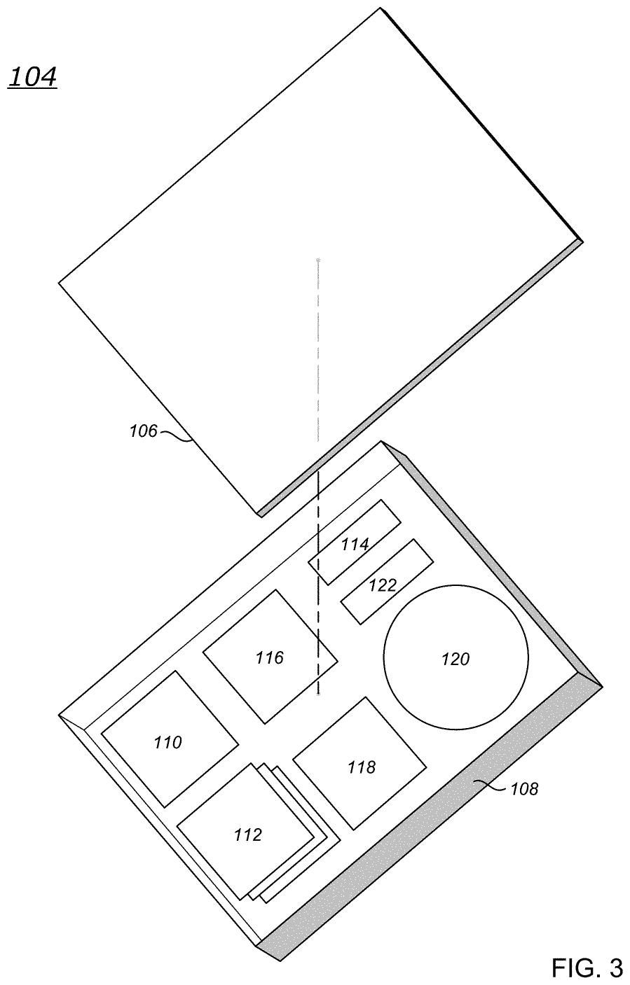

FIG. 3 is an electronics module 104 embodiment of a sleep apnea detection device 100. The electronics module 104 embodiment includes a processor 110, one or more memory devices 112, an input/output (I/O) port 114, at least one micro-electromechanical system (MEMs) device 116, a transceiver 118, and a power supply circuit that includes at least one power storage device 120 and at least one power control circuit 122. Other operative components of the electronics module 104 are not shown for brevity and to avoid obscuring the inventive teaching herein. The operative components of the electronics module 104 are communicatively coupled (e.g., one or more address buses, data buses, and other such conduits that conform to any selected protocol) and electrically coupled (e.g., one or more power buses, power planes, and the like) as known by one of ordinary skill in the art.

In at least some cases, the processor 110 is a low power microcontroller and the one or more memory devices 112 include volatile memory (e.g., random access memory (RAM)) and non-volatile memory (e.g., read only memory (ROM), flash memory, or the like). In at least some cases, computer readable software instructions stored in the one or more memory devices 112 are executed by the processor 110 to carry out the functions of the sleep apnea detection device 100.

The MEMs device 116 may include plurality of MEMs devices drawn from a group that includes one or more of an accelerometer, a microphone, a motion sensor, a gyroscope, a pressure sensor, a thermal actuator, a magnetic actuator, a high aspect electrostatic resonator, a comb-drive, or some other MEMs structures. The transceiver 118 in at least some embodiments is arranged for wireless, bidirectional communications with at least one computing device such as computing device 16 (FIG. 1).

The power storage device 120 in some cases is a battery. The battery may supply any needed current at any determined voltage. In at least one case, the power storage device 120 is arranged to provide 1.8 VDC at 100 milliAmp hours (100 mAH). In other embodiments, the power storage device 120 is arranged to provide current at 150 mAH, 200 mAH, or some other power (e.g., voltage, current, time) rating. The power storage device 120 may be a rechargeable battery, a non-rechargeable battery, a capacitor, or some other storage device. A power control circuit 122 may be arranged as a recharge circuit electronically coupled to the power storage device 120. In addition, or in the alternative, the power control circuit 122 is arranged to deliver power to the processor 110, memory 112, MEMs devices 116, transceiver 118, and other circuits of the electronics module 104. In at least one case, the power control circuit 122 includes an induction circuit arranged to receive a wireless power signal and further arranged to charge the power storage device 120 based on the received wireless power signal.

In at least some cases, the I/O port 114 works cooperatively with the power control circuit 122 and the power storage device 120. In these and other cases, the I/O port 114 may be used to pass a wired power supply signal into the electronics module 104, and the wired power supply signal can be used to charge the power storage device 120 (e.g., a battery). In some cases, the I/O port 114 may be used to: 1) pass information to the electronics module 104, 2) pass information from the electronics module 104, or 3) pass information both to and from the electronics module 104. The I/O port 114 may communicate via a single wire protocol (SWP), a multi wire protocol (e.g., USB), or via some other physical and communication protocol. In at least some cases, the I/O port is useful for retrieving sleep apnea patient data from the electronics module 104.

The I/O port 114 may be used in other ways in some embodiments. For example, in at least some cases, the I/O port 114 is useful for uploading computing instructions (e.g., software, firmware, or the like), control parameters, patient data, or still other information to the electronics module 104. In at least one case, control information from a user or a computing device will direct the sleep apnea detection device 100 to form an Apnea Hypopnea Index according to a selected technique.

In at least some cases, the I/O port 114 is also configured with at least one light-emitting diode (LED) and at least one photo-diode. In these embodiments, the LED and photo-diode may cooperate to cooperatively illuminate a portion of the patient's blood through the patient's skin and detect reflected or otherwise dispersed light caused by the illumination. Such an arrangement may be used by the sleep apnea detection device 100 to determine the patient's oxygen saturation level.

In FIG. 3, the electronics module 104 is arranged inside a housing having a first part 108 and a second part 106. The first part 108 of the housing is arranged to contain active electronics circuitry, and the second part 106 of the housing is arranged to cover the first part 108 of the housing. Other embodiments are contemplated. For example, in some cases, the electronic module 104 is encased in a silicone rubber. In some cases, the electronics module is encased in a conformal coating that is applied as a bath, a spray, a deposition, or in some other technique.

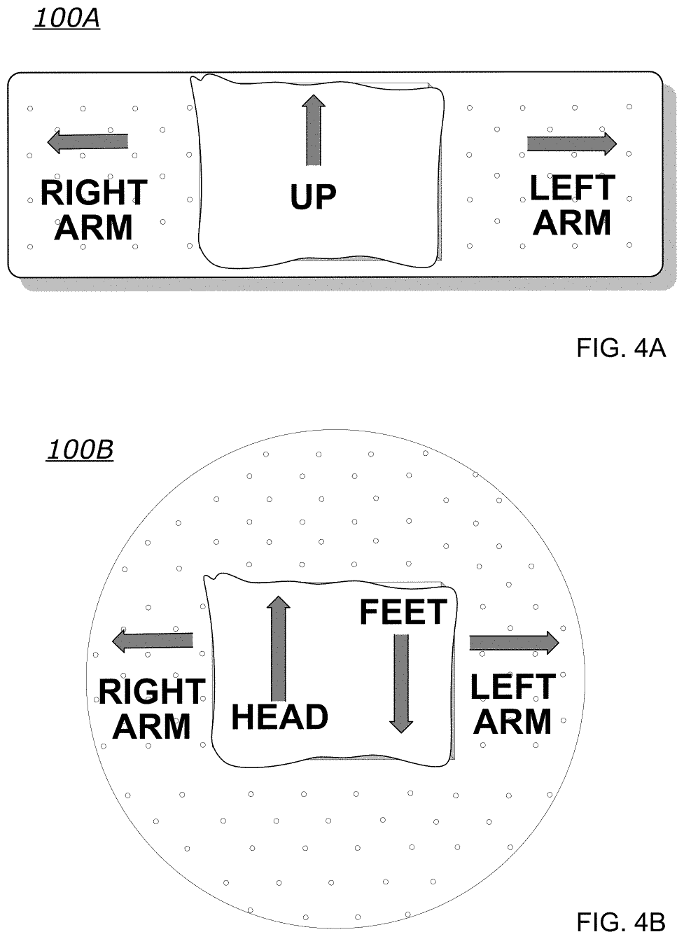

FIGS. 4A and 4B are sleep apnea detection device 100A, 100B embodiments, respectively. In the present disclosure, FIGS. 4A-4B may be collectively referred to as FIG. 4. In the present disclosure, each of the sleep apnea detection devices, individually or collectively, may be referred to herein as sleep apnea detection device 100 unless the context dictates otherwise.

The embodiments of FIG. 4 include optional fiducial markings to help orient the sleep apnea detection device 100 on the patient. The sleep apnea detection devices may be arranged for placement on the torso, head, neck, or some other location on the patient. In at least some cases, the sleep apnea detection device 100 does not need any particular orientation. In such cases, for example, MEMs device 116 may include a multi-axis accelerometer that can provide sufficient output signaling that a patient's sleep orientation can be determined. In these or other cases, for example, MEMs device 116 may include a directional microphone or a vibration sensor that can isolate a direction from which snoring originates, and this directional information can alternatively or additionally be used to determine a patient's sleep orientation. Other circuit-based arrangements are also contemplated. In at least some other embodiments, the sleep apnea detection devices 100 may be arranged as a pendant, an article of clothing (e.g., a nightshirt, a gown, a hat, or the like), a wrist-worn device, or in some other form factor.

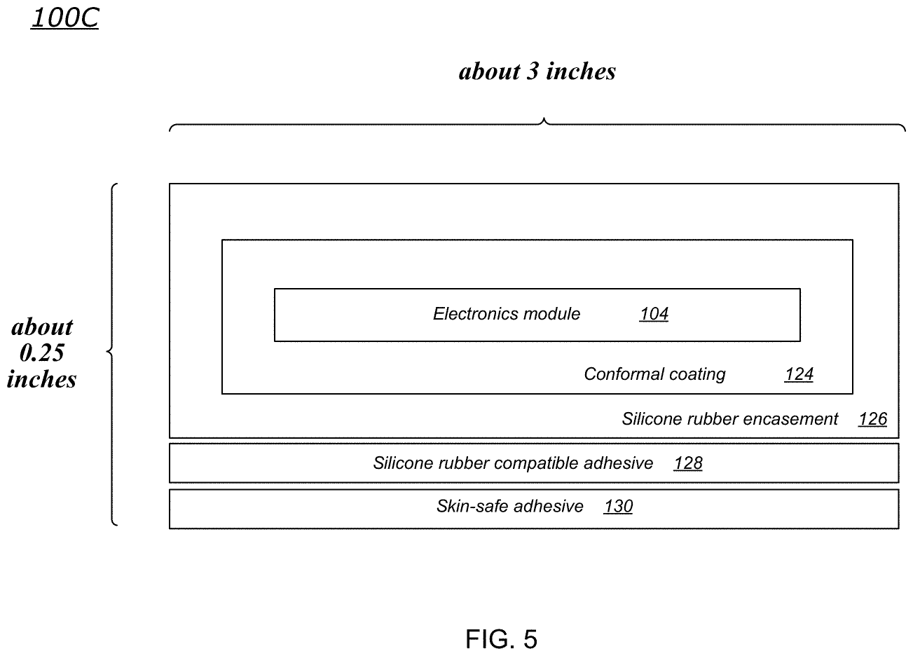

FIG. 5 is a right side, cross-sectional view of yet one more sleep apnea detection device 100C embodiment. In the present disclosure, each of the sleep apnea detection devices, individually or collectively, may be referred to herein as sleep apnea detection device 100 unless the context dictates otherwise. A top view of the sleep apnea detection device 100D of FIG. 5 (not shown) may show the sleep apnea detection device 100 as having a square shape, a rectangular shape, a round shape, a triangular shape, or as having any other desirable regular or irregular shape. In the embodiment of FIG. 5, the sleep apnea detection device 100 has a height of about 0.25 inches and at least one linear dimension of about three inches. As shown in FIG. 5, the electronics module 104 is sealed with a conformal coating 124, and the coated module is covered via a silicone rubber encasement 126. A particular silicone rubber-based compatible adhesive 120 is applied to at least one surface of the silicone rubber encasement 126, and a particular skin-safe adhesive 130 is applied to the at least one surface of the silicone rubber compatible adhesive 128. As so constructed, the electronics module 104 of the sleep apnea detection device 100 is well-separated from the body of the patient.

Systems that employ teaching of the sleep apnea detection device 100 described herein reduce or resolve many known issues related to the accurate determination of sleep apnea. The sleep apnea detection device 100 works in real time to measure a patient's breathing pattern, level of snoring, and current sleep position.

In at least some embodiments, the one sleep apnea detection device 100, or a plurality of sleep apnea detection devices 100 working cooperatively as a system, is further arranged to measure certain brain signals of the patient. When so measured, the certain brain signals are analyzed to determine which of a plurality of specific types of sleep apnea a patient may be suffering from. For example, in various cases, the sleep apnea detection device 100 is used to determine that a patient has obstructive sleep apnea (OSA), central sleep apnea (CSA), or mixed sleep apnea (MSA). In at least some cases where a type of sleep apnea is determined, at least one sleep apnea detection device 100 is temporarily affixed to the patient's head (e.g., forehead, neck, crown, or some other area).

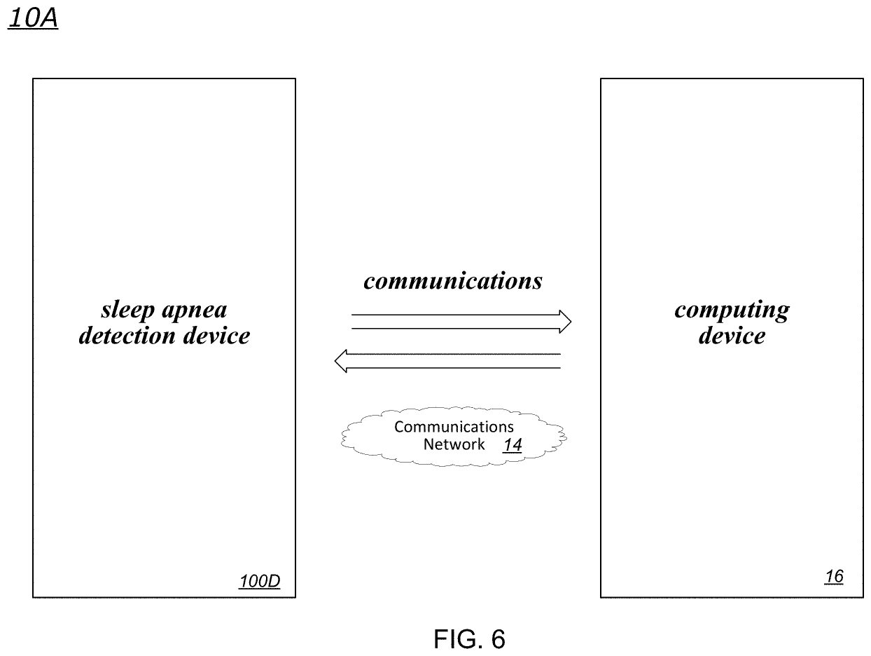

FIG. 6 is a system 10A that communicatively couples a sleep apnea detection device 100D, which is any type of sleep apnea detection device 100 taught in the present disclosure, to a computing device 16. The two devices in FIG. 6 may communicate via a wired or wireless medium in accordance with a communications network 14. In the system 10A of FIG. 6, the wireless communications are bidirectional, but in other cases, the communications may be unidirectional. In at least some cases, a transceiver 118 of the sleep apnea detection device 100 is a BLUETOOTH LOW ENERGY compatible transceiver and the communications are bidirectional and wireless.

In use, the system 10A of FIG. 6 is deployed when a medical practitioner or a patient seeks to determine if the patient is suffering from sleep apnea. The sleep apnea detection device 100 is affixed to the abdomen of the patient, and the sleep apnea detection device 100 is communicatively coupled to the computing device 16. The patient then goes to sleep.

As the patient sleeps, a MEMs device 116 formed as a digital microphone captures information related to the patient's breathing pattern. A same or different MEMs device 116 formed as a digital microphone captures information related to the patient's snoring. Yet one more MEMs device 116 formed as an accelerometer (e.g., a three axis accelerometer arranged to capture Cartesian coordinate information in three planes: an x-plane, a y-plane, and a z-plane) detects and captures information in association with motion of the patient's diaphragm when the patient breathes. Alternatively, or in addition, the MEMs device 116 formed as the accelerometer or a different MEM's device 116 formed as a different accelerometer may produce information used to determine the patient's sleep position.

The processor 110 of the sleep apnea detection device 100 includes a time circuit (e.g., a clock circuit) or works operatively with a time circuit to coordinate all of the captured information in accordance with a common time base. In this way, the accelerometer-based (e.g., breathing, sleep position) information may be cooperatively analyzed with audio-based (e.g., snoring, breathing) information that occurs at a same time.

In at least one case of the system 10A of FIG. 6, the power storage circuit 120 of the sleep apnea detection device 100 supplies about 1.8 VDC for at least eight hours, and the sleep apnea detection device 100 consumes under 20 mA when in operation. In this implementation, the sleep apnea detection device 100 may be constantly transmitting raw data according to a given schedule via the transceiver 118, which is formed as a BLUETOOTH LOW ENERGY device.

In at least one other case of the system 10A, the sleep apnea detection device 100 constantly captures data according to a given schedule, and the raw data, or processed data, is stored in the one or more memory devices 112 while the patient sleeps (e.g., two to ten hours, less than two hours, more than ten hours, or some other time period). After the patient wakes, or upon a determined trigger event, the sleep apnea detection device 100 is arranged to communicate the accumulated data to the computing device 16.

Processing of raw data produced by the one or more MEMs devices 116 may occur in the sleep apnea detection device 100, the computing device 16, or both the sleep apnea detection device 100 and the computing device 16 in any suitable duplicative or cooperative combination.

One technique used to determine whether or not a patient has sleep apnea is to calculate an Apnea Hypopnea Index (i.e., an AHI score). The AHI score is one mechanism used by a medical practitioner to quantify the number of sleep apnea induced pauses in a patient's breathing as the patient sleeps. There are a number of ways to calculate an AHI score.

In one technique, an AHI score is calculated using just the patient's breathing patterns. In another technique, the patient's breathing patterns and the patient's oxygen saturation level are used to calculate an AHI score. In these or other techniques, the patient's breathing patterns and brain waves are analyzed to determine if a breathing pause is truly sleep apnea.

For the sake of efficient description herein, an exemplary technique to calculate an AHI score is based on the patient's breathing patterns. As described herein, other techniques are contemplated. In the present embodiment, the patient's breathing patterns are analyzed, and the AHI score is calculated by determining how many ten-second pauses in the patient's breathing occur during each hour. An average of all of the calculated hourly AHI scores is determined to obtain the average AHI score for any given period of sleep.

In at least one embodiment of system 10A, when the sleep apnea detection device 100 is in operation, at least one MEMs device 116 is formed as a digital accelerometer in which the Z axis is aligned in an anterior-posterior direction. Stated differently, when the patient is lying prostrate, the Z axis will be realized in a floor-to-ceiling direction. In the embodiment, the digital accelerometer is used to detect breath movements by calculating a 100-point running average of Z axis measurements. A sample rate (e.g., a measurement frequency) of 104 samples per second is selected, but other rates are contemplated. An accelerometer range of +/−2 g, and a maximum resolution of 4096 values are selected, but other ranges and resolutions are also contemplated. The inventor has learned that such a configuration provides a sufficient signal-to-noise ratio that allows breathing to be detected.

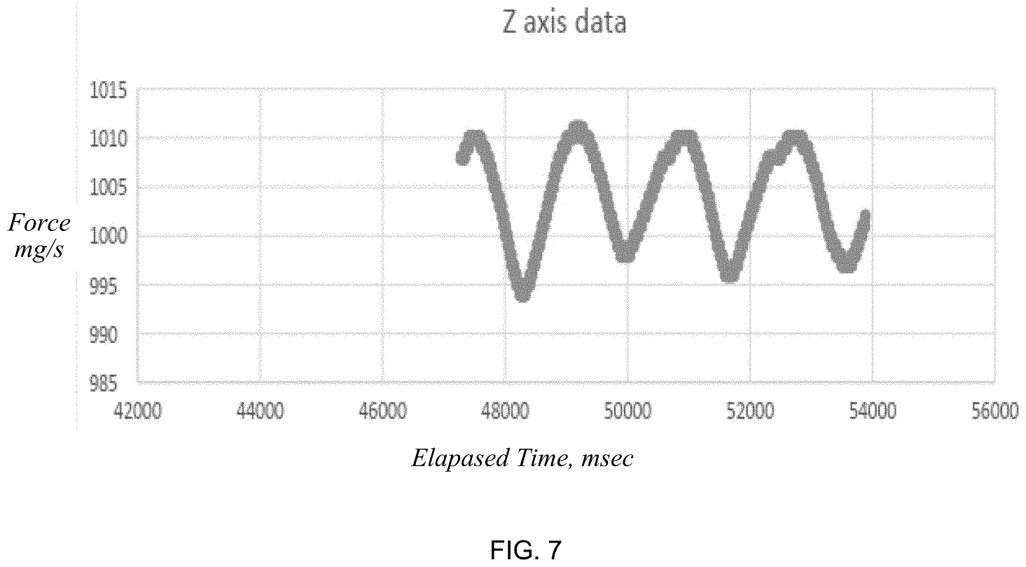

FIG. 7 is a graph representing accelerometer data of a 48 year old man breathing in a supine position on a bed. In the graph, the horizontal axis of the graph represents an elapsed time in milliseconds (msec) since a point where data capture began, and the vertical axis shows smoothed accelerometer force data in milli-G per second (mg/s). From this data, the AHI score can be calculated by determining, for each 60 minute period, how many pauses in inhales occur. In this case, a pause may be defined as the absence of an inhale for more than 10 seconds. In the graph of FIG. 7, a perfect AHI score of 0.0 is determined. In the graph of FIG. 7, between 48000 msec and 53000 msec, three inhales can be seen. In other cases, where an otherwise detectable inhale is absent, a different AHI score will be determined.

In these and other embodiments, human breath detection can be detected in other ways using the sleep apnea detection device 100. For example, because a MEMs device 116 formed as an accelerometer can detect a static direction of gravity when the device is affixed directly to the patient's abdomen, the real time sleeping position of the patient can be determined with a great deal of accuracy. When the patient is in a supine position (i.e., lying generally face up, on his back), for example, the Z-axis will read about 1000 mg/s (i.e., about one G per second (1 g/s)), and force measurements in both the X-axis and the Y-axis force measurements will be close to zero. If the patient is in the prone position (i.e., lying generally face down, on his stomach), the Z-axis will read about minus 1000 mg/s (i.e., about minus one G per second (−1 g/s)), and the X-axis and the Y-axis force measurements will be close to zero. By calculating the static gravitational force, a compass-like position can be determined for any possible angle that the patient sleeps in, in real time, and this data may be coordinated with the real time breath determination values. In this way, a medical practitioner may help a patient remediate sleep apnea by changing their sleep habits.

In addition, in the alternative, or even in these same embodiments, a MEMs device 116 can be formed as a digital microphone, a vibration detection device (e.g., motion detector, accelerometer, or the like), or some other device in the sleep apnea detection device 100. In this way, the sleep apnea detection device 100 can be used to better detect the patient's breathing, snoring, or breathing and snoring. The patient's snoring can be detected, for example, by looking for vibration patterns in the sound wave at specific frequencies. By so tuning the data collection, other types of night-time sounds such as alarms, fans, heating units, barking dogs, sirens, and the like can be filtered out. One useful result of a MEMs device 116 formed in this way is a binary data output that indicates, in real time, whether or not snoring is occurring during the a time window of any desirable duration (e.g., in the previous 10 seconds, in the previous 20 seconds; a threshold number of times in the last 30 minutes, or the like).

By reliable calculation of an AHI score, a medical practitioner, or even an operating algorithm, can determine whether or not a patient suffers from sleep apnea. The sleep apnea detection device 100 described herein is small, portable, easily worn, and likely to provide suitably acceptable results in patients of all ages, genders, sizes, physical conditions, and the like. Patients that are determined to suffer from sleep apnea may seek appropriate medical care from a qualified medical practitioner.

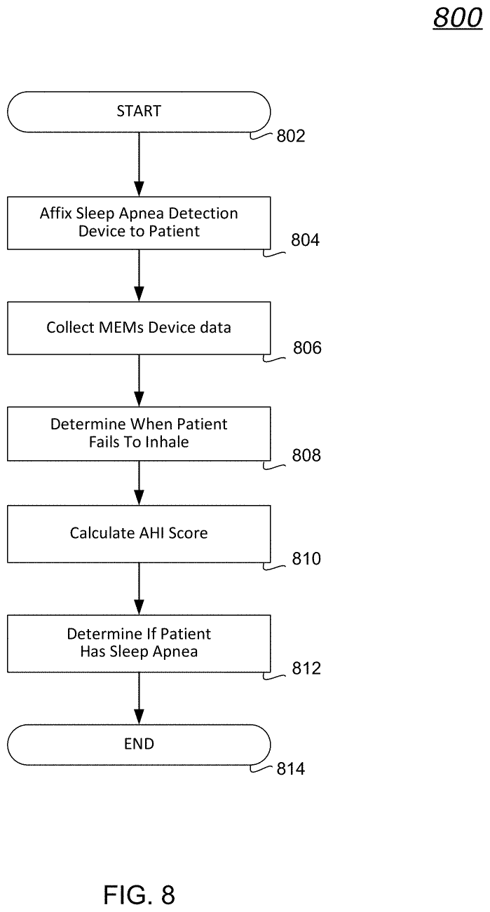

FIG. 8 is a data flow diagram representing a sleep apnea detection process 800 carried out with a sleep apnea detection device embodiment.

At 802, the procedure begins. The procedure is implemented with an embodiment of a particular sleep apnea detection device embodiment. Among other things, the sleep apnea detection device will have a processor such as a microcontroller, at least one memory (e.g., volatile memory, non-volatile memory, or volatile and non-volatile memory), any number of micro-electromechanical system (MEMs) devices drawn from a group of MEMs devices that includes one or more of an accelerometer, a microphone, a motion sensor, a gyroscope, a pressure sensor, a thermal actuator, a magnetic actuator, a high aspect electrostatic resonator, and a comb-drive. The sleep apnea detection device will also typically include at least one wired or wireless transceiver such as a BLUETOOTH LOW ENERGY transceiver, and at least one wired or wireless power source to power the sleep apnea detection device. In at least some cases, the sleep apnea detection device is arranged in a housing (e.g., a silicone rubber encasement, a plastic or nylon compartment, or the like), arranged on a flexible substrate (e.g., a thin tape), encased in a conformal coating, or suitably arranged in some other way.

The power source of the sleep apnea detection device is in some cases a battery, and the power source may be rechargeable. For example, the sleep apnea detection device may be a battery or some other storage device that is arranged to deliver at least 1.8 VDC for 8 hours and additionally or alternatively, at least 20 milliamps (mA) over 8 hours.

The sleep apnea detection device may be arranged as an adhesive bandage or a thin tape having the identified electronic components integrated thereon. In some cases, the sleep apnea detection device is arranged as a bracelet, a pendant, a mask, a wearable article of clothing (e.g., a hat, a scarf, a headband, a night shirt, a gown, a glove, or some other article), or in some other form factor. In at least one case, the sleep apnea detection device is a mobile computing device such as a smartphone, a smart watch, a handheld computer, or some other such computing device. In at least one case, the sleep apnea detection device is arranged as an Internet of Things (IoT) device.

In some cases, the sleep apnea detection device is sized at about one inch by about three inches by about one quarter inch. In some cases, the sleep apnea detection device is sized at about three quarters of a cubic inch or less. In at least one case, the sleep apnea detection device is less than four inches long in a first linear dimension and less than one inch deep in a second dimension, wherein the first linear dimension is orthogonal to the second linear dimension. Other sizes, dimensions, and volumes are contemplated.

In some cases, the sleep apnea detection device is affixed to a patient via a skin-safe adhesive to thereby allow secure overnight placement of the sleep apnea detection device and reasonably easy removal.

At 804, the sleep apnea detection device is affixed to a patient. In some cases, the affixation is achieved via a skin-safe adhesive. In other cases, the affixation is achieved via placement of the sleep apnea detection device in a wrist band, a hat, a scarf, a night shirt, or via some other means. In some cases, the sleep apnea detection device is affixed to the patient's abdomen, and in other cases, the sleep apnea detection device is affixed to the patient's forehead, neck, wrist, or some other portion on or in proximity to the patient's body. After the sleep apnea detection device is positioned in the appropriate location, the patient will enter a period of sleep, and processing falls to 806.

At 806, the one or more MEMs devices of the sleep apnea detection device will begin to collect data. The collected data will indicate the patient's breathing (e.g., measure the patient's breathing or breathing pattern). In some cases, the sleep apnea detection device is arranged on or in proximity to the patient's skull (e.g., the sleep apnea detection device is affixed to the patient's forehead), and the collected data includes measured brain signals. The measured brain signals can in some cases be used to determine if a patient's sleep apnea is obstructive sleep apnea (OSA), central sleep apnea (CSA), mixed sleep apnea (MSA), or some other kind of sleep apnea.

In addition to measuring the patient's breathing or pattern of breathing, one or more MEMs devices may be arranged to measure snoring information such as the audio volume, audio frequency pattern, vibration, duration, the patient's sleep position, and still other kinds of data. When the patient's sleep position is determined, such as using one or more MEMs devices arranged as an accelerometer, the determined sleep positions may be a determination of whether the patient is lying on his back, lying on his belly, lying on his right side, lying on his left side, lying in a fetal position, or in some other position. For example, in some cases, the “z-axis” of a suitably arranged accelerometer will register about one G of force when the patient is on his back (i.e., supine) or about minus one G of force when the patient is lying on his stomach (i.e., prone). Additionally, if the “x-axis” is identified as arm-to-arm and if the “y-axis” is identified as chin-to-belly, then the x-axis will register about one G of force when the patient is on his left side, and the x-axis will register about minus one G when the patient is on his right side. In at least some embodiments, the static gravitational force data from the MEMs devices (e.g., accelerometer data) can be used to calculate a compass-like position for any angle that the patient sleeps in at any given time during the sleep apnea detection session. In at least one case, the sleep apnea detection device will have at least one fiducial marking arranged to orient the sleep apnea detection device on the patient.

In at least one embodiment, the sleep apnea detection device includes at least one input/output (I/O) port arranged to determine oxygen saturation level in the patient's blood.

After data is collected at 806 by the at least one MEMs device, processing falls to 808.

At 808, the sleep apnea detection device will determine when a patient fails to exhale. And at 810, the sleep apnea detection device will calculate an Apnea Hypopnea Index (AHI) score.

The determination of when a patient fails to inhale is made using data collected by one or more MEMs devices. In at least some cases, the patient's inhalations are determined, and in these or in other cases, the patient's exhalations are determined. Inhalation and exhalations, once such breathing patterns are detected, can be used to determine when the patient fails to inhale over a determined first period of time.

In some cases, a determination of when a patient fails to inhale, which may be identified as “paused breathing,” is a period of ten or more continuous seconds without the patient taking an inhaling breath. In some cases, a determination of an AHI score is based on the number of paused breathing events (i.e., the number of times the patient failed to inhale for at least ten seconds) that occur in an hour. In these or other cases, the AHI score is an average of all determined hourly AHI scores that occurred during a sleep session. The sleep session may be between about two hours and ten hours, less than one hour, less than 16 hours, or some other duration.

In some cases, the MEMs device arranged as a digital, breath-detection accelerometer is operated at a sample rate of between 20 and 200 samples per second. In some cases, the MEMs device arranged as the digital, breath-detection accelerometer has a resolution of at least 12-bits.

In some cases, the AHI score is calculated using only the number of paused breathing events (i.e., the patient fails to inhale for a given first amount of time) that occur over a second time period. In some cases, the AHI score is calculated using the patient's breathing pattern as just described in addition to an oxygen saturation level in the patient's blood. In these cases, one or more I/O ports in the sleep apnea detection device may be used with a light source and a light detection means to determine blood saturation levels. In still other cases, the AHI score is calculated using the patient's breathing pattern and one or more of the patient's brain wave patterns (e.g., a skull-based sensor is arranged to receive brain wave signaling). In some cases, the patient's breathing pattern is coordinated with the patient's sleeping position to calculate a sleeping position-based AHI score. In some cases, the patient's breathing pattern is coordinated with the patient's snoring to calculate a snoring-based AHI score. In one or more of these cases, snoring may be determined with a MEMs based microphone, a MEMs based frequency detection scheme, a MEMs based vibration detection scheme, or in some other way. In these cases, the scheme may be arranged to distinguish the breathing, snoring, or breathing and snoring from other noise (e.g., fans, alarms, sirens, animal noises, and the like), and the non-breathing and non-snoring noise may be filtered out. In at least one case, a determination of “snoring” is formed as a binary signal output based over a time-window of a determined duration (e.g., five seconds, ten seconds, or some other duration).

In at least one case, the one or more memories of the sleep apnea detection device are arranged to store a plurality of AHI score calculation algorithms. A user input, a computing device input, a programmatic input, or some other parameter is received to select which of the plurality of AHI score calculation algorithms is used to determine the AHI score.

After the AHI score is calculated, processing falls to 812.

At 812, it is determined whether or not the patient has sleep apnea. In at least some cases, the determination of sleep apnea is based on the AHI score crossing a determined threshold. For example, an AHI score may be determined based on how many paused breathing events (i.e., determining when the patient fails to inhale over a determined first period of time) that occur over a second period of time. If the first period of time is ten seconds, and the second period of time is one hour, a determination of sleep apnea may be based on a threshold of three events, five events, eight events, fifteen events, or some other number of events. That is, if the patient fails to inhale for at least ten seconds more than a first selected number of times (e.g., three times, five times, or some other first selected number of times), on average, per hour, then the patient may be determined to have mild sleep apnea. If the patient fails to inhale for at least ten seconds more than a second selected number of times (e.g., ten times, fifteen times, or some other second selected number of times), on average, per hour, then the patient may be determined to have moderate or severe sleep apnea. Other time periods, threshold numbers of paused breathing events, and determinations of severity of sleep apnea are of course contemplated. In at least some cases, the other time periods, threshold numbers of paused breathing events, and determinations of severity of sleep apnea may be determined by a medical practitioner. In at least some cases, these parameters may be manually entered, programmatically entered, or set in some other way.

In some cases, the determination of some or all calculations and algorithms is performed in the sleep apnea detection device. In some cases, the determination of some or all calculations and algorithms is performed in a different computing device from the sleep apnea detection device. In still other cases, the determination of some or all calculations and algorithms is shared between the sleep apnea detection device and another computing device. In any or all of these cases, the sleep apnea detection device may transmit data to the other computing device. The data may be communicated in a wired way or a wireless way. The data may be communicated as raw data, interim data, or final data. That is, the sleep apnea detection device may communicate raw MEMs based data to the other computing device, partially processed data to the other computing device, or a determination of sleep apnea data to the other computing device. In some cases, the sleep apnea detection device communicates data as it is collected, in real time. In other cases, the sleep apnea detection device accumulates data and communicates the accumulated data at a later, determined time to the other computing device. In some cases, the sleep apnea detection device communicates data to a plurality of other computing devices.

In some cases, data is communicated between the sleep apnea detection device and another computing device via a wireless BLUETOOTH LOW ENERGY protocol.

In at least one case, the sleep apnea detection device transmits data indicating the patient's breathing, via the at least one transceiver, to another computing device, and the other computing device determines whether the patient has sleep apnea.

In at least one case, the sleep apnea detection device transmits a determination that the patient has sleep apnea, via the at least one transceiver, to another computing device, and the other computing device outputs an indication of the determination that the patient has sleep apnea.

After the determination and processing indicating whether or not a patient has sleep apnea at 812, processing falls to 814.

Processing ends at 814.

Having now set forth certain embodiments, further clarification of certain terms used herein may be helpful to provide a more complete understanding of that which is considered inventive in the present disclosure.

In the embodiments of present disclosure, one or more particular electronic structures of the sleep apnea detection device are coupled, connected, or otherwise arranged in cooperation. The various components and devices of the embodiments are interchangeably described herein as “coupled,” “connected,” “attached,” and the like. It is recognized that once assembled, the system is suitably arranged to perform the teaching described herein. The materials and the junctions formed at the point where two or more structures meet in the present embodiments are sealed to a mechanically, medically, or otherwise industrially acceptable level.

FIG. 8 includes a data flow diagram illustrating a non-limiting process that may be used by embodiments of a sleep apnea detection device 100. In this regard, each described process may represent a module, segment, or portion of software code, which comprises one or more executable instructions for implementing the specified logical function(s). It should also be noted that in some implementations, the functions noted in the process may occur in a different order, may include additional functions, may occur concurrently, and/or may be omitted.

The figures in the present disclosure illustrate portions of one or more non-limiting computing device embodiments such as one or more components of the sleep apnea detection device 100. The computing devices may include operative hardware found in conventional computing device apparatuses such as one or more processors, volatile and non-volatile memory, serial and parallel input/output (I/O) circuitry compliant with various standards and protocols, wired and/or wireless networking circuitry (e.g., a communications transceiver), one or more user interface (UI) modules, logic, and other electronic circuitry.

Processing devices, or “processors,” as described herein, include central processing units (CPU's), microcontrollers (MCU), digital signal processors (DSP), application specific integrated circuits (ASIC), peripheral interface controllers (PIC), state machines, and the like. Accordingly, a processor as described herein includes any device, system, or part thereof that controls at least one operation, and such a device may be implemented in hardware, firmware, or software, or some combination of at least two of the same. The functionality associated with any particular processor may be centralized or distributed, whether locally or remotely. Processors may interchangeably refer to any type of electronic control circuitry configured to execute programmed software instructions. The programmed instructions may be high-level software instructions, compiled software instructions, assembly-language software instructions, object code, binary code, micro-code, or the like. The programmed instructions may reside in internal or external memory or may be hard-coded as a state machine or set of control signals. According to methods and devices referenced herein, one or more embodiments describe software executable by the processor, which when executed, carries out one or more of the method acts.

As known by one skilled in the art, a computing device has one or more memories, and each memory comprises any combination of volatile and non-volatile computer-readable media for reading and writing. Volatile computer-readable media includes, for example, random access memory (RAM). Non-volatile computer-readable media includes, for example, read only memory (ROM), magnetic media such as a hard-disk, an optical disk, a flash memory device, a CD-ROM, and/or the like. In some cases, a particular memory is separated virtually or physically into separate areas, such as a first memory, a second memory, a third memory, etc. In these cases, it is understood that the different divisions of memory may be in different devices or embodied in a single memory. The memory in some cases is a non-transitory computer medium configured to store software instructions arranged to be executed by a processor. Some or all of the stored contents of a memory may include software instructions executable by a processing device to carry out one or more particular acts.

The computing devices illustrated herein (e.g., sleep apnea detection device 100, computing device 16, and the like) may further include operative software found in a conventional computing device such as an operating system or task loop, software drivers to direct operations through I/O circuitry, networking circuitry, and other peripheral component circuitry. In addition, the computing devices may include operative application software such as network software for communicating with other computing devices, database software for building and maintaining databases, and task management software where appropriate for distributing the communication and/or operational workload amongst various processors. In some cases, the computing device is a single hardware machine having at least some of the hardware and software listed herein, and in other cases, the computing device is a networked collection of hardware and software machines working together in a server farm to execute the functions of one or more embodiments described herein. Some aspects of the conventional hardware and software of the computing device are not shown in the figures for simplicity.

Amongst other things, the exemplary computing devices of the present disclosure (e.g., sleep apnea detection device 100, computing device 16) may be configured in any type of mobile or stationary computing device such as a remote cloud computer, a computing server, a smartphone, a tablet, a laptop computer, a wearable device (e.g., eyeglasses, jacket, shirt, pants, socks, shoes, other clothing, hat, helmet, other headwear, wristwatch, bracelet, pendant, other jewelry), or the like. Accordingly, the computing devices include other components and circuitry that is not illustrated, such as, for example, a display, a network interface, memory, one or more central processors, camera interfaces, audio interfaces, and other input/output interfaces. In some cases, the exemplary computing devices may also be configured in a different type of low-power device such as a headboard mounted multimedia device, an Internet-of-Things (IoT) device, a multimedia device, a motion detection device, or some other device.

When so arranged as described herein, each computing device may be transformed from a generic and unspecific computing device to a combination device arranged comprising hardware and software configured for a specific and particular purpose such as to provide a determined technical solution. When so arranged as described herein, to the extent that any of the inventive concepts described herein are found by a body of competent adjudication to be subsumed in an abstract idea, the ordered combination of elements and limitations are expressly presented to provide a requisite inventive concept by transforming the abstract idea into a tangible and concrete practical application of that abstract idea.

The embodiments described herein use computerized technology to improve the technology of sleep apnea detection, but other techniques and tools remain available to determine if a patient has sleep apnea. Therefore, the claimed subject matter does not foreclose the whole or even substantial sleep apnea detection technological area. The innovation described herein uses both new and known building blocks combined in new and useful ways along with other structures and limitations to create something more than has heretofore been conventionally known. The embodiments improve on computing systems which, when un-programmed or differently programmed, cannot perform or provide the specific sleep apnea detection system features claimed herein. The embodiments described in the present disclosure improve upon known sleep apnea detection processes and techniques. The computerized acts described in the embodiments herein are not purely conventional and are not well understood. Instead, the acts are new to the industry. Furthermore, the combination of acts as described in conjunction with the present embodiments provides new information, motivation, and business results that are not already present when the acts are considered separately. There is no prevailing, accepted definition for what constitutes an abstract idea. To the extent the concepts discussed in the present disclosure may be considered abstract, the claims present significantly more tangible, practical, and concrete applications of said allegedly abstract concepts. And said claims also improve previously known computer-based systems that perform sleep apnea detection operations.

Software may include a fully executable software program, a simple configuration data file, a link to additional directions, or any combination of known software types. When a computing device updates software, the update may be small or large. For example, in some cases, a computing device downloads a small configuration data file as part of software, and in other cases, a computing device completely replaces most or all of the present software on itself or another computing device with a fresh version. In some cases, software, data, or software and data is encrypted, encoded, and/or otherwise compressed for reasons that include security, privacy, data transfer speed, data cost, or the like.

Database structures, if any are present in the sleep apnea detection devices 100 described herein, may be formed in a single database or multiple databases. In some cases hardware or software storage repositories are shared amongst various functions of the particular system or systems to which they are associated. A database may be formed as part of a local system or local area network. Alternatively, or in addition, a database may be formed remotely, such as within a distributed “cloud” computing system, which would be accessible via a wide area network or some other network.

Input/output (I/O) circuitry and user interface (UI) modules include serial ports, parallel ports, universal serial bus (USB) ports, IEEE 802.11 transceivers, BLUETOOTH and BLUETOOTH LOW ENERGY transceivers, and other transceivers compliant with protocols administered by one or more standard-setting bodies, displays, projectors, printers, keyboards, computer mice, microphones, micro-electromechanical (MEMS) devices such as accelerometers, and the like.

In at least one embodiment, devices such as the sleep apnea detection device 100 may communicate with other computing devices 16 via communication over a communications network 14. The network may involve an Internet connection or some other type of local area network (LAN) or wide area network (WAN). Non-limiting examples of structures that enable or form parts of a network include, but are not limited to, an Ethernet, twisted pair Ethernet, digital subscriber loop (DSL) devices, wireless LAN, Wi-Fi, Worldwide Interoperability for Microwave Access (WiMax), or the like. The communications network 14 may alternatively or additionally involve a personal area network (PAN) that includes wired and wireless short range communications arranged according to any selected protocol such as BLUETOOTH, BLUETOOTH LOW ENERGY (BLE), IEEE 802.11 (WiFi), universal serial bus (USB), and the like.

In the present disclosure, memory may be used in one configuration or another. The memory may be configured to store data. In the alternative or in addition, the memory may be a non-transitory computer readable medium (CRM). The CRM is configured to store computing instructions executable by a processor of the sleep apnea detection device 100. The computing instructions may be stored individually or as groups of instructions in files. The files may include functions, services, libraries, and the like. The files may include one or more computer programs or may be part of a larger computer program. Alternatively or in addition, each file may include data or other computational support material useful to carry out the computing functions of a sleep apnea detection device 100.

Buttons, keypads, computer mice, memory cards, serial ports, bio-sensor readers, touch screens, and the like may individually or in cooperation be useful to a medical practitioner or patient operating the sleep apnea detection device 100. The devices may, for example, input control information into the system. Displays, printers, memory cards, LED indicators, temperature sensors, audio devices (e.g., speakers, piezo device, etc.), vibrators, and the like are all useful to present output information to the medical practitioner or patient operating the sleep apnea detection device 100. In some cases, the input and output devices are directly coupled to the sleep apnea detection device 100 and electronically coupled to a processor or other operative circuitry. In other cases, the input and output devices pass information via one or more communication ports (e.g., RS-232, RS-485, infrared, USB, etc.).

As described herein, for simplicity, a medical practitioner and a patient may in some cases be described in the context of the male gender. It is understood that a medical practitioner and a patient can be of any gender, and the terms “he,” “his,” and the like as used herein are to be interpreted broadly inclusive of all known gender definitions. As the context may require in this disclosure, except as the context may dictate otherwise, the singular shall mean the plural and vice versa; all pronouns shall mean and include the person, entity, firm or corporation to which they relate; and the masculine shall mean the feminine and vice versa.

The terms, “real-time” or “real time,” as used herein and in the claims that follow, are not intended to imply instantaneous processing, transmission, reception, or otherwise as the case may be. Instead, the terms, “real-time” and “real time” imply that the activity occurs over an acceptably short period of time (e.g., over a period of microseconds or milliseconds), and that the activity may be performed on an ongoing basis (e.g., receiving MEMs data, determining paused breathing events, determining sleep apnea, and the like). An example of an activity that is not real-time is one that occurs over an extended period of time (e.g., hours or days) or that occurs based on intervention or direction by a medical practitioner or patient or other activity.

In the absence of any specific clarification related to an express use in a particular context, where the terms “substantial” or “about” in any grammatical form are used as modifiers in the present disclosure and any appended claims (e.g., to modify a structure, a dimension, a time, a measurement, or some other characteristic), it is understood that the characteristic may vary by up to 30 percent. For example, a sleep apnea detection device 100 may be described as being having a particular linear dimension of about three inches. In these cases, a sleep apnea detection device 100 that is formed having a particular linear dimension of exactly three inches is implied. And though different from the exact precision of the term, the use of “about” to modify the characteristic permits a variance of the dimension by up to 30 percent. Accordingly, a sleep apnea detection device 100 that is formed having a particular linear dimension of 2.1 inches is “about three inches,” and a sleep apnea detection device 100 that is formed having a particular linear dimension of 3.9 inches is also “about three inches.” In contrast, a sleep apnea detection device 100 that is formed having a particular linear dimension of one inch or five inches is not “about three inches.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, and this concept is also encompassed within the invention, subject to any specifically excluded limit in the stated range. Where the stated range includes one or both of the limits, ranges excluding either or both of those included limits are also included in the invention.

Unless defined otherwise, the technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

In the present disclosure, when an element (e.g., component, circuit, device, apparatus, structure, layer, material, or the like) is referred to as being “on,” “coupled to,” or “connected to” another element, the elements can be directly on, directly coupled to, or directly connected to each other, or intervening elements may be present. In contrast, when an element is referred to as being “directly on,” “directly coupled to,” or “directly connected to” another element, there are no intervening elements present.

The terms “include” and “comprise” as well as derivatives and variations thereof, in all of their syntactic contexts, are to be construed without limitation in an open, inclusive sense, (e.g., “including, but not limited to”). The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present disclosure, the terms first, second, etc., may be used to describe various elements, however, these elements are not limited by these terms unless the context clearly requires such limitation. These terms are only used to distinguish one element from another. For example, a first machine could be termed a second machine, and, similarly, a second machine could be termed a first machine, without departing from the scope of the inventive concept.

The singular forms “a,” “an,” and “the” in the present disclosure include plural referents unless the content and context clearly dictates otherwise. The conjunctive terms, “and” and “or” are generally employed in the broadest sense to include “and/or” unless the content and context clearly dictates inclusivity or exclusivity as the case may be. The composition of “and” and “or” when recited herein as “and/or” encompasses an embodiment that includes all of the elements associated thereto and at least one more alternative embodiment that includes fewer than all of the elements associated thereto.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list.

The headings and Abstract of the Disclosure provided herein are for convenience only and do not interpret the scope or meaning of the embodiments.

The teaching of sleep apnea detection devices 100 in the present disclosure provide several technical effects and advances to the field of sleep apnea detection.

The various embodiments described above can be combined to provide further embodiments. Various features of the embodiments are optional, and, features of one embodiment may be suitably combined with other embodiments. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method to detect sleep apnea, comprising:
affixing a sleep apnea detection device to a patient, the sleep apnea detection device having a processor, at least one memory, at least one micro-electromechanical system (MEMs) device, at least one transceiver, and at least one power source to power the sleep apnea detection device;
collecting data with the at least one MEMs device that indicates the patient's breathing;
based on the collected data, determining at least one of the patient's inhalations and the patient's exhalations;
determining when the patient fails to inhale over a determined first period of time;
based on a number times the patient fails to inhale, calculating an Apnea Hypopnea Index (AHI) score; and
based on the AHI score crossing a determined threshold, determining that the patient has sleep apnea.

2. The method of claim 1, wherein the at least one MEMs device includes a digital accelerometer, a digital microphone, and a digital motion detector.

3. The method of claim 1, comprising:
transmitting the data that indicates the patient's breathing, via the at least one transceiver, to a computing device; and
determining that the patient has sleep apnea via the computing device.

4. The method of claim 1, comprising:
transmitting the determination that the patient has sleep apnea, via the at least one transceiver, to a computing device; and
outputting from the computing device an indication of the determination that the patient has sleep apnea.

5. The method of claim 1, wherein the determined first period of time is about 10 seconds.

6. The method of claim 1, comprising:
collecting data with the at least one MEMs device data that indicates the patient's level of snoring; and
coordinating the patient's indicated level of snoring with the determination of when the patient fails to inhale.

7. The method of claim 6, wherein the at least one MEMs device includes a digital accelerometer to detect a vibration level associated with the patient's level of snoring.

8. The method of claim 6, wherein the at least one MEMs device includes a digital accelerometer to detect a frequency of vibration associated with the patient's level of snoring.

9. The method of claim 1, comprising:
collecting data with the at least one MEMs device data that indicates the patient's current sleep position; and
coordinating the patient's indicated sleep position with the determination of when the patient fails to inhale.

10. The method of claim 9, wherein the at least one MEMs device includes a digital accelerometer to detect when the patient is lying prone, supine, on a left side, or on a right side.

11. A sleep apnea detection device, comprising:
a processor,
at least one memory communicatively coupled to the processor;
at least one micro-electromechanical system (MEMs) device arranged to produce digital data representing sleep information that is processed by the processor;
at least one transceiver, and
at least one power source to power the processor, at least one memory, at least one MEMs device, and at least one transceiver, wherein the processor is arranged to execute computer instructions stored in the at least one memory that, when executed by the processor, cause the processor to:
collect a patient's breathing data with the at least one MEMs device;
determine, based on the collected breathing data at least one of the patient's inhalations and the patient exhalations;
determine when the patient fails to inhale over a determined first period of time;
calculate an Apnea Hypopnea Index (AHI) score based on the patient's failure to inhale; and
determine that the patient has sleep apnea based on the AHI score.

12. The sleep apnea detection device of claim 11, comprising:
a conformal coating over the processor, the at least one memory, the at least one MEMs device, the at least one transceiver, and the at least one power source.

13. The sleep apnea detection device of claim 12, comprising:
a silicone-rubber based material encasing the processor, the at least one memory, the at least one MEMs device, the at least one transceiver, and the at least one power source.

14. The sleep apnea detection device of claim 11, comprising:
a flexible substrate having supported thereon the processor, the at least one memory, the at least one MEMs device, the at least one transceiver, and the at least one power source; and
a skin-sensitive adhesive applied to at least one portion of the flexible substrate.

15. The sleep apnea detection device of claim 11, wherein the sleep apnea detection device is less than four inches long in a first linear dimension and less than one inch deep in a second dimension, the first linear dimension orthogonal to the second linear dimension.

16. The sleep apnea detection device of claim 11, wherein the sleep apnea detection device is less than one cubic inch in volume.

17. The sleep apnea detection device of claim 11, wherein the sleep transceiver is arranged according to a BLUETOOTH LOW ENERGY protocol.

18. The sleep apnea detection device of claim 11, comprising:
at least one fiducial marking arranged to orient the sleep apnea detection device on the patient.

19. A non-transitory computer-readable storage medium whose stored contents configure a computing system to perform a method, the method comprising:
receiving data collected with the at least one micro-electromechanical system (MEMs) device that indicates a patient's breathing, the at least one MEMs device formed in a sleep apnea detection device affixed to the patient;
based on the collected data, determining when the patient fails to inhale over a determined first period of time; and
based on a number of times in a determined second period of time that the patient fails to inhale over the determined first period of time, determining that the patient has sleep apnea.

20. The non-transitory computer-readable storage medium according to claim 19 whose stored contents configure the computing system to perform the method, the method further comprising:
coordinating the patient's sleep position with the data that indicates the patient's breathing.
